(19) United States
(12) Patent Application Publication
(10) Pub. No.: US 2012/0289524 A1
(43) Pub. Date: Nov. 15, 2012

(54) PYRROLO [3,2-E] [1,2,4] TRIAZOLO [1,5-A] PYRIMIDINES DERIVATIVES AS INHIBITORS OF MICROGLIA ACTIVATION

(75) Inventors: David Scopes, Cambridgeshire (GB); David Horwell, Cambridgeshire (GB)

(73) Assignee: Senexis Limited, Cambridgeshire (GB)

(21) Appl. No.: 13/501,027

(22) PCT Filed: Oct. 7, 2010
(86) PCT No.: PCT/EP2010/065002

§ 371 Date: Jul. 26, 2012

(30) Foreign Application Priority Data
Oct. 9, 2009 (GB) 0917774.2

(51) Int. Cl. A61K 31/519; A61P 25/28; C07D 471/14

(52) U.S. Cl. 514/267; 544/251

(57) ABSTRACT

The invention relates to novel compounds useful in the treatment and prophylaxis of disease. Compounds of the formula (I):

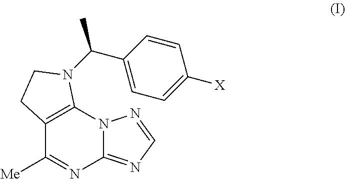

wherein X is halogen, independently selected form chlorine and fluorine and their pharmaceutically acceptable salts are useful in the treatment and prophylaxis of diseases caused by activation of microglia, particularly Alzheimer's disease.

This invention relates to novel compounds useful in the treatment and prophylaxis of disease. Particularly the current invention provides compounds useful in the treatment and prophylaxis of diseases caused by activation of microglia, particularly where the activation is caused by amyloid proteins such as β amyloid.

In the development of pharmaceutically active compounds, the provision of compounds with improved activity (e.g. at the target site, or in model systems) is important. However, it is also important that active compounds have useful pharmacokinetic, pharmacodynamic and toxicological properties. For example high compound bioavailability means that less of the compound needs to be used to achieve a given blood level. Particularly improved oral bioavailability means that oral dosage forms are more effective. Consequently, improvements in activity at the target may be balanced against other properties such as bioavailability, and in vivo half life which are also important.

EP1433480 discloses the use of certain pyrimidine derivatives for the treatment of central nervous system diseases. Uryu et al (2002) Brain Research, 946(2), 298-306 and Uryu et al (2003) Biochem. Biophys. Res. Com., 303(1), 302-305, both discuss RS-1178, a compound recited in EP1433480. U.S. Pat. No. 4,007,189 describes Pyrrolotriazolopyrimidine derivatives which are said to be useful as antihypertensive agents. JP 52116497 describes triazolopyrimidines said to be useful as vasodilators and antihypertensives, Y. Sato et al., J. Med. Chem. (1980), 23, 927-937 describes 1,2,4-triazolo[1,5-a]pyrimidines fused to heterocyclic systems, which are said to be useful as vasodilators. EP347252 describes triazolo- and pyrazolopyrrolopyrimidines useful in the treatment of cachexia.

The current invention provides specific, novel compounds of the formula (I), that are not disclosed in EP1433480, that are potent inhibitors of the activation of macrophages, and that are useful as pharmaceutical actives in the treatment of disease and which have improved activity over previously disclosed compounds.

Thus a first aspect of the invention provides a compound of the formula (I) or a pharmaceutically acceptable salt thereof:

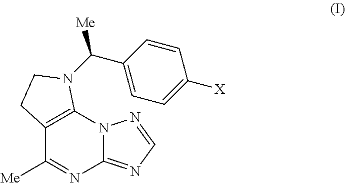

wherein X is halogen, independently selected form chlorine and fluorine of which Fluorine is preferred. Preferred pharmaceutically acceptable salts include those formed with strong acids such as hydrochloric acid, sulfuric acid, phosphoric acid, methanesulfonic acid, benzene sulfonic acid and particularly hydrochloric acid and methanesulfonic acid.

A second embodiment of the invention provides the use of a compound of the formula (I) or a pharmaceutically acceptable salt thereof, in therapy.

Compounds of the formula (I) are potent inhibitors of the activation of macrophages in vitro, via a pathway that differs from that by which lipopolysaccharides and zymosan act (EP1433480). This system is used as a model for microglial activation (Uryu et al (2002) Brain Research, 946(2), 298-306). The compounds of the invention are therefore useful in conditions in which microglial activation plays a role. Microglial activation has been proposed in a number of mammalian neurodegenerative conditions, particularly in Alzheimer's disease, Parkinson's disease (e.g. Teisman and Schulz 2004), Huntington's chorea (e.g. Bonifati and Kishore 2006) and Pick's disease (e.g. Schofield et al 2003). Compounds of the formula (I) have particularly been demonstrated to be inhibitors of macrophage activation by amyloid protein and so are particularly useful in conditions in which activation is induced by amyloid proteins, particularly in Parkinson's disease and Alzheimer's disease.

Compounds of the invention may be used without further components to the composition, that is to say that the composition consists essentially of the compound of the invention, but will generally be used as a pharmaceutically acceptable composition, which optionally comprises one or more pharmaceutically acceptable carriers or diluents. The compounds will generally be provided in a composition that is sterile and pyrogen free.

Preparations suitable for any of the commonly used routes of administration such as oral, rectal, nasal, topical or perenteral may be prepared by methods well known in the art of pharmacy. These may take the form of solutions, suspensions, tablets, pills, capsules, powders, sustained release formulations and the like.

Suitable doses of the compounds of the invention will be in the range 0.1 mg of compound per kg body weight to 100 mg/kg, preferably 1 mg/kg to 100 mg/kg and more preferably 1 mg/kg to 10 mg/kg.

A third embodiment of the invention provides a pharmaceutical composition comprising a compound of the formula (I) or a pharmaceutically acceptable salt thereof, preferably in combination with a pharmaceutically acceptable carrier or diluent.

In addition, the compounds of the invention may be administered with one or more additional therapeutic compounds. For example, one or more anti-inflammatory compounds (eg NSAIDS), which have been shown to slow the onset of neurodegenerative diseases; one or more compounds suitable for the treatment of Alzheimer's disease (eg beta-amyloid aggregation inhibitors, gamma-secretase inhibitors, gamma-secretase modulators or beta-secretase inhibitors);or compounds for the treatment of Parkinson's disease. Thus, the pharmaceutical formulations of the invention can additionally comprise such compounds. However, it is of course possible to administer such compounds separately, either at the same time as a compound of the invention or sequentially.

Thus, in a fourth aspect, the present invention provides a composition comprising a compound of the invention, together with one or more additional therapeutic compounds for simultaneous, sequential or separate use. The one or more additional compounds can be chosen from the examples discussed above.

A fifth aspect of the invention provides a method of treatment of diseases involving the activation of microglia (particularly where microglia are activated by amyloid protein), comprising administering to a patient in need thereof, a therapeutically effective amount of a compound of the formula (I) or a pharmaceutically acceptable salt thereof.

A sixth aspect of the invention provides the use of a compound of the formula (I) or a pharmaceutically acceptable salt thereof in the manufacture of a medicament for the treatment of diseases involving the activation of microglia (particularly where microglia are activated by amyloid protein).

A seventh aspect of the invention provides a compound of the formula (I) or a pharmaceutically acceptable salt thereof for the treatment of diseases involving the activation of microglia (particularly where microglia are activated by amyloid protein).

The current invention will now be described with the help of the following examples, schemes and figures. Further embodiments within the scope of the invention will become apparent to the skilled worker in the light of these.

### SYNTHETIC EXAMPLE

### (S)-8-[1-(4-Fluorophenypethyl]-5-methyl-7,8-dihydro-6H-pyrrolo [3,2e][1,2,4]triazolo[1,5-a]pyrimidine [Compound 1]

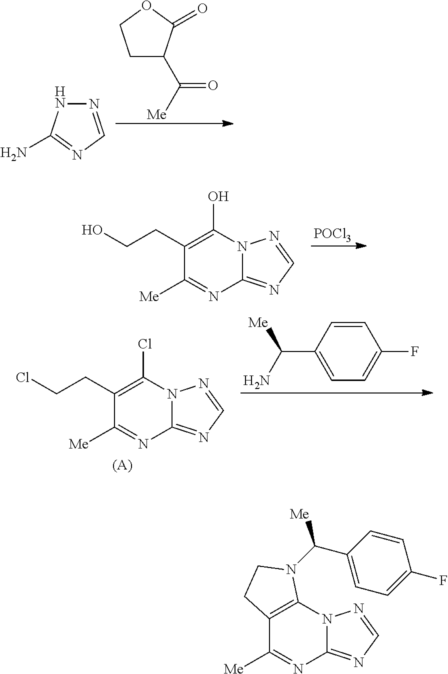

Intermediate (A) may be synthesised by the method of Y. Sato et al., J. Med. Chem. (1980) 23, 927-937.

1. 6-(2-Hydroxyethyl)-5-methyl[1,2,4] triazolo [1,5-a]pyrimidin-7(4H)-one

A mixture of 3-aminotriazole (350 g, 4.1 mol) and α-acetyl-γ-butyrolactone (524.8 g, 4.1 mol) was stirred in IMS (2.8L) and BF₃.Et₂O (85 mL, 0.6 mol) added over 15 min. After 3 days stirring at ambient temperature the solid was collected by filtration and dried on the filter.

¹H NMR (d6-DMSO) δ 13.6 (1H, br), 10.37 (1H, s), 8.40 (1H, s), 4.30 (2H, t), 2.88 (2H, t) and 2.50 (3H, s).

The solid was stirred in water (1.7L) and triethylamine (422 mL, 4.1 mol) added, after which the solid dissolved. After stirring for 2 days at ambient temperature, acetic acid (1 eq. 90 mL) was added. The mixture was stirred for 1 h and the solid filtered, dried on the filter and under vacuum at 40° C. for 4 h to give the dihydroxy compound as a white solid, 430 g, 2.2 mol (54%).

¹H NMR (d6-DMSO) δ 8.16 (1H, s), 3.48 (2H, t), 2.62 (2H, t) and 2.36 (3H,s). ES⁺195 (100%), M+H⁺.

2. 7-Chloro-6-(2-chloroethyl)-5-methyl[1,2,4]triazolo[1,5-a]pyrimidine

POCl₃ (130 mL) was added to 6-(2-hydroxyethyl)-5-methyl[1,2,4] triazolo [1,5-a]pyrimidin-7(4H)-one (111 g, 0.57 mol) in a single portion (generates an exotherm) and the mixture stirred and heated in 40° C. steps to 120° C. (at 70-80° C. all the solids dissolved). After 5 h heating was stopped and the mixture allowed to cool overnight. Some residual POCl₃ was removed under vacuum and the residue added to well-stirred water (1L) over 40 min. The temperature rose upon addition and ice was added periodically to keep the temperature below 25° C., care being taken to avoid the gum settling below the water. The mixture was cooled in an ice-bath, stirred and the pH adjusted to approximately 7 with aqueous ammonia solution and the solid collected. The solid was taken into dichloromethane (150 mL), the separated water removed, any solids removed by filtration and the organic solution dried over MgSO₄. After concentration, the crude material was purified by elution under vacuum through silica (eluent: 1.5-2% methanol/dichloromethane) to give the dichloro compound as a white solid (62 g, 0.27 mol).

¹H NMR (d6-DMSO) δ 8.66 (1H, s), 3.90 (2H, t), 3.33 (2H, t) and 2.75 (3H,s). ES⁺231 (100%), M+H⁺.

3. (S)-8-[1-(4-Fluorophenypethyl]-5-methyl-7,8-dihydro-6H-pyrrolo [3,2e] [1,2,4]triazolo[1,5-a]pyrimidine A mixture of 7-chloro-6-(2-chloroethyl)-5-methyl[1,2,4]triazolo[1,5-a]pyrimidine (390 mg, 1.7 mmol), (S)-4-fluoro-α-methylbenzylamine (378 mg, 2.7 mmol) and sodium carbonate (324 mg, 3.0 mmol) in ethanol (5 mL) was heated under reflux for 5 h. The mixture was cooled to room temperature, filtered, and the solvent removed from the filtrate. The crude product was triturated with di-isopropylether to afford 8-[(S)-1-(4-fluorophenyl)ethyl]-5-methyl-7,8-dihydro-6H-pyrrolo[3,2e][1,2,4] triazolo[1,5-a]pyrimidine as a yellow solid (430 mg, 85%).

¹H NMR (CDCl₃) δ 1.71 (3 H, d), 2.39 (3 H, s), 3.06 (2 H, m), 3.44 (1 H, m), 3.84 (1 H, m), 6.85 (1 H, q), 7.03 (2 H, m), 7.34 (2 H, m) and 8.30 (1 H, s). LCMS (ES⁺): 298 (MH⁺, 100%).

### COMPARATIVE EXAMPLE

### (S)-8-[1-(Phenypethyl]-5-methyl-7,8-dihydro-6H-pyrrolo[3,2e][1,2,4] triazolo[1,5-a]pyrimidine [Compound 3]

This compound is the (S)-enantiomer of RS-1178 [Compound 2], previously discussed in Uryu et al (2002) Brain Research, 946(2), 298-306 and Uryu et al (2003) Biochem. Biophys. Res. Com., 303(1), 302-305. General synthetic routes to this compound are disclosed in Sato et al., J. Med. Chem. (1980), 23, 927-937. The compound was prepared by the same method as used for the preparation of (S)-8-[1-(4-fluorophenyl)ethyl]-5-methyl-7,8-dihydro-6H-pyrrolo[3,2e][1,2,4]-triazolo [1,5-a]pyrimidine, except that (S)-α-methylbenzylamine was used in place of (S)-4-fluoro-α-methylbenzylamine.

¹H NMR (CDCl₃) δ 1.71 (3 H, d), 2.35 (3 H, s), 3.10 (2 H, m), 3.50 (1 H, m), 4.00 (1 H, m), 6.73 (1 H, q), 7.30-7.45 (5 H, m) and 8.48 (1 H, s). MS (ES⁺): 280 (MH⁺, 100%).

The (R) enantiomer [Compound 4] is prepared analogously using the (R)-α-methylbenzylamine.

### EXPERIMENTAL EXAMPLES

1. Inhibition of β-amyloid Induced Activation.

Mouse BALB/c monocyte macrophages, J774.2, {ECACC 85011428} were grown and sub-cultured in cell media (DMEM containing 10% FBS, 1% L-glutamine and 1% penicillin/streptomycin). The J774 cells were plated at 100,000 cells/well in 50 μl cell media on 96 well plates and placed in a 37° C., 5% CO2 incubator overnight prior to experiments.

The addition of Aβ(1-42) and compounds to the J774 cells was performed using a Biotek precision 2000 liquid handling instrument. 3 μl of compound in DMSO ranging from 8 μM to 6 mM were pipetted into a “daughter plate” containing 294 μl of cell media and mixed thoroughly. 3 μl of Aβ(1-42) in DMSO at 4 mM was then added to the “daughter plate” and mixed thoroughly. 50 μl was then removed from the “daughter plate” and added to the plated J774 cells. The final concentrations in the wells containing 100 μl cell media were 20 μM Aβ(1-42), the compounds ranged from ˜40 nM to 30 μM in 1% DMSO and also in the presence of 50 U/ml Interferon gamma. The plates were incubated for 24 hours in a 37° C., 5% CO2 incubator. After 24 hours incubation the media from the wells were collected and stored at −20° C. until required for testing.

The nitric oxide levels in the media were tested using the Griess assay (Promega G2930) using the manufacturer's instructions.

The TNF-alpha levels in the media were tested using a TNF-alpha ELISA (R&D Systems MTA00) or Meso Scale Discovery MS6000 Mouse Proinflammatory-7 kit, using the manufacturers instructions.

TABLE 1

| Com- | | IC₅₀ (μM) | IC₅₀ (μM) |
|---|---|---|---|
| pound | Compound | NO release | TNF-α release |
| 1 | 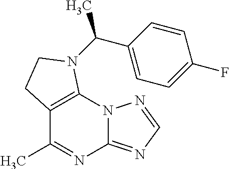 | 0.24 | 0.48 |
| 2 | 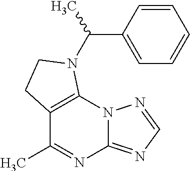 | 3 | 5.5 |
| 3 | 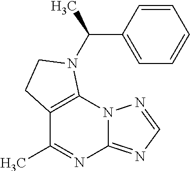 | 1.5 | 2.3 |
| 4 | 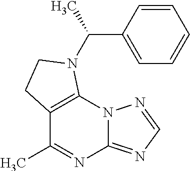 | 30 | >30 |

TABLE 2
Pharmacokinetic data

| | T_{1/2} | T_{1/2} | Oral bio- |
|---|---|---|---|
| Compound | per oral | i.v. | availability |
| 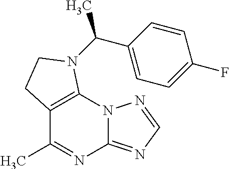 | 3.2 | 0.87 | 98.5 |
| 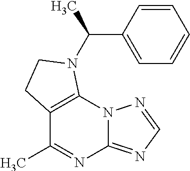 | 2.4 | 0.22 | 34.5 |

The compounds of the invention therefore have improved ½ life and bioavailability compared to other compounds of the class

1. A compound of the formula (I) or the pharmaceutically acceptable salt there of 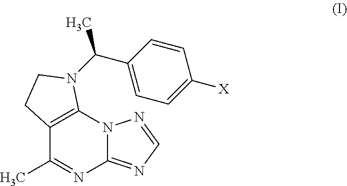
wherein
X is selected from fluorine and chlorine.

2. A compound or the pharmaceutically acceptable salt thereof according to claim 1 wherein X is fluorine.

3. A pharmaceutical composition comprising a compound of the formula (I) or a pharmaceutically acceptable salt thereof in combination with a pharmaceutically acceptable carrier or diluent.

4. A pharmaceutical composition as claimed in claim 3 which further comprises one or more compounds selected from anti-inflammatory compounds (eg NSAIDS), compounds suitable for the treatment of Alzheimer's disease (eg beta-amyloid aggregation inhibitors, gamma-secretase inhibitors, gamma-secretese modulators or beta-secretase inhibitors) or compounds for the treatment of Parkinson's disease.

5. A composition comprising a compound according to claim 1, together with one or more additional therapeutic compounds for simultaneous, sequential separate use.

6. A composition as claimed n claim 5 wherein the additional therapeutic compound is selected from anti-inflammatory compounds (eg NSAIDS), compounds suitable for the treatment of Alzheimer's disease (eg beta-amyloid aggregation inhibitors, gamma-secretase inhibitors, gamma-secretase modulators or beta-secretase inhibitors) or compounds for the treatment of Parkinson's disaease.

7. A method of using in therapy comprising administering a compound of the formula (1) or a pharmaceutically acceptable salt thereof according to claim 1.

8. A method of treatment of a disease involving the activation of microglia, comprising administering to a patient in need thereof, a therapeutically effective amount of a compound of the formula (I) or a pharmaceutically acceptable salt thereof according to claim 1.

9. A method of treatment according to claim 8, wherein the disease involving the activation of microglia is Alzheimer's disease.

10. A method for the treatment of a disease involving the activation of microglia comprising administering a medicament comprising a compound of the formula (I) or a pharmaceutically acceptable salt thereof according to claim 1.

11. The method according to claim 10 wherein the disease involving the activation of microglia is Alzheimer's disease.

12. A compound of the formula (I) or a pharmaceutically acceptable salt thereof according to claim 1 for the treatment of diseases involving the activation of microglia.

13. A compound of the formula (I) or a pharmaceutically acceptable salt thereof according to claim 1 for the treatment of Alzheimer's disease.
